(19) United States
(12) Patent Application Publication
(10) Pub. No.: US 2025/0160655 A1
(43) Pub. Date: May 22, 2025

(54) VIBRO-ACOUSTIC MODELING OF CARDIAC ACTIVITY

(71) Applicant: CARDIOSOUNDS LLC, Ann Arbor, MI (US)

(72) Inventor: Kevin Wittrup, Ann Arbor, MI (US)

(21) Appl. No.: 18/838,960

(22) PCT Filed: Feb. 14, 2023
(86) PCT No.: PCT/US2023/062524

§ 371 Date: Aug. 15, 2024

Related U.S. Application Data
(60) Provisional application No. 63/310,501, filed on Feb. 15, 2022.

(51) Int. Cl. A61B 5/02; A61B 5/00; A61B 5/021; A61B 5/11; A61B 5/282; A61B 5/308; G16H 50/20; G16H 50/70

(52) U.S. Cl. CPC A61B 5/02028; A61B 5/02141; A61B 5/1102; A61B 5/282; A61B 5/308; A61B 5/6823; A61B 5/726; A61B 5/7282; A61B 5/7289; G16H 50/20; G16H 50/70; A61B 2560/045; A61B 2560/0468

(57) ABSTRACT

Systems and methods assess cardiac function based on isolating components of a vibrational signature which are attributable to heart valve closure and bulk movement of a heart during each cardiac cycle.

### TECHNICAL FIELD

The present disclosure relates generally to systems and methods for vibro-acoustic-based modeling of cardiac activity.

### BACKGROUND

Sensors are used in the medical industry to assess the cardiovascular health of a user or human subject. They are used in the management of patient health. Some devices monitor and store health data to assist monitoring patient condition and managing the progression of an illness.

### SUMMARY

This specification describes methods and systems for vibro-acoustic based modeling of cardiac activity. These methods and systems efficiently sense and relay cardiac vibro-acoustic signatures to a signal processing module to extract signal features. The signal features are analyzed to predict cardiovascular features (e.g., systolic blood pressure or diastolic blood pressure, heart failure, heart disease, acute cardiopulmonary system failure, myocardial infarction, congestive heart failure, or cardiopulmonary collapse).

Using the cardiac vibro-acoustic signatures as indicators of cardiac activity is advantageous because cardiac vibro-acoustic signatures indicate cardiovascular features that are difficult to detect through other methods. The vibro-acoustic signature contains information that is indicative of the mechanical pumping function of the heart and can be measured non-invasively using specialized approaches as outlined herein that do not require the types of expensive medical diagnostic equipment currently used for characterizing and monitoring cardiac function.

In an aspect, an apparatus includes a body that is attachable non-permanently and non-invasively to a human subject's chest, at least two vibration sensing elements contained in the body, wherein the vibration sensing elements measure a vibrational signature on a chest wall of the subject, wherein a first vibration sensing element is indexed from a xiphoid of the subject and a second vibration sensing element is located in or between a second and third intercostal space of the subject, and at least two electrodes contained within the body, the at least two electrodes electrically attachable to skin of the subject and a corresponding amplifier circuit to acquire an ECG signal, wherein the apparatus is configured to synchronously acquire signals from the chest wall of the subject and transmit said signals to an external data collection device, wherein an external data collection device monitors the signals, wherein the signals are indicative of a hemodynamic state of the subject and provide diagnostic decision support related to one or more of: blood pressure, heart valve function, acute cardiopulmonary system failure, and chronic heart conditions.

In some embodiments, acute cardiopulmonary system failure includes one or more of myocardial infarction, congestive heart failure, or cardiopulmonary collapse.

In some embodiments, chronic heart conditions include hypertension.

In some embodiments, the apparatus includes a third vibration sensing element located in or between the second and third intercostal space of the subject.

In some embodiments, the body includes multiple arms, each arm having a different length, wherein one of the vibration sensing elements is contained within each arm.

In an aspect, a method of assessing cardiac activity includes isolating components of the vibrational signature which are attributable to heart valve closure and bulk movement of a heart during each cardiac cycle, with reference to features of the ECG signal, such that mechanical and electrical activity of the cardiac cycle is simultaneously comprehended, and such that signal features indicative of cardiopulmonary state are extracted from the combined signal set, wherein extracted signal features and methods include one or more of: timing, duration, and intensity of vibroacoustic cardiac signals relative to each other and ECG waveform features, frequency content of vibroacoustic cardiac signals in a 10 Hertz to 200 Hertz frequency range, energy levels in a subset of wavelet packets determined uniquely for each subject based on signals of the subject, which are combined with observations from other subjects and states with similar attributes to include a signal phenotype, a time of arrival of signal wavelet packets at each sensor, the wavelets being from separate heart signatures, to normalize for signal transfer function between source and receiver that is different for each individual, and high resolution time-frequency characterization of heart signatures using maximum overlap wavelet transforms such that the signature of valve flutter upon closure is characterized.

In some embodiments, the method for operating on the processed signals and signal features includes training individual models for single individuals in a training set over short periods of time to create a reference library of models associated with a signal phenotype, and combining individual reference models with an abstract feature extracted from a multi-layer convolutional neural network as inputs to a prediction model, wherein new observations from previously unseen subjects are compared to pre-existing signal phenotypes to select a subset of reference models from the reference library that best match the new observations, such that a new prediction is made by presenting new data to a selected subset of reference models and determining a consensus output weighted by a relative similarity of each model to the new signal phenotype.

In some embodiments, the model is a random forest, neural network, or support vector machine model.

In some embodiments, the method includes receiving the components of the vibrational signature which are attributable to heart valve closure and bulk movement of the heart from at least two vibration sensing elements, wherein the vibration sensing elements measure a vibrational signature on a chest wall of a subject, wherein a first vibration sensing element is indexed from a xiphoid of the subject and a second vibration sensing element is located in or between a second and third intercostal space of the subject.

In some embodiments, the method includes receiving the components of the vibrational signature which are attributable to heart valve closure and bulk movement of the heart from at least a third vibration sensing element, the third vibration sensing element located in or between the second and third intercostal space of the subject.

In some embodiments, the method includes receiving the ECG waveform features from at least two electrodes electrically attachable to skin of the subject and a corresponding amplifier circuit to acquire the ECG waveform features.

### DESCRIPTION OF DRAWINGS

FIGS. 1A-D illustrate a cardiac sensor.

FIG. 2 illustrates a cardiac sensor.

FIG. 3 illustrates a cardiac sensor.

FIG. 4 is a block diagram of a system including a cardiac sensor.

FIGS. 5A and 5B are illustrations of extracted signal features.

FIGS. 6A-F are illustrations of extracted signal features.

FIG. 7 is a block diagram of a model architecture for training a model.

FIG. 8 is a block diagram of a model architecture for analyzing signal features of a user.

### DETAILED DESCRIPTION

This specification describes methods and systems for vibroacoustic-based modeling of cardiac activity. These methods and systems efficiently sense and relay cardiac vibro-acoustic signatures to a signal processing module to extract signal features. The signal features are analyzed to predict cardiovascular features or the cardiopulmonary state (e.g., systolic blood pressure or diastolic blood pressure, fluid responsiveness, degree of congestive heart failure, etc.) of a patient. Information about the cardiopulmonary state of a patient or user are helpful to someone providing medical care to the patient. Awareness of cardiovascular status over time provides critical information affecting medical care and the health of the patient.

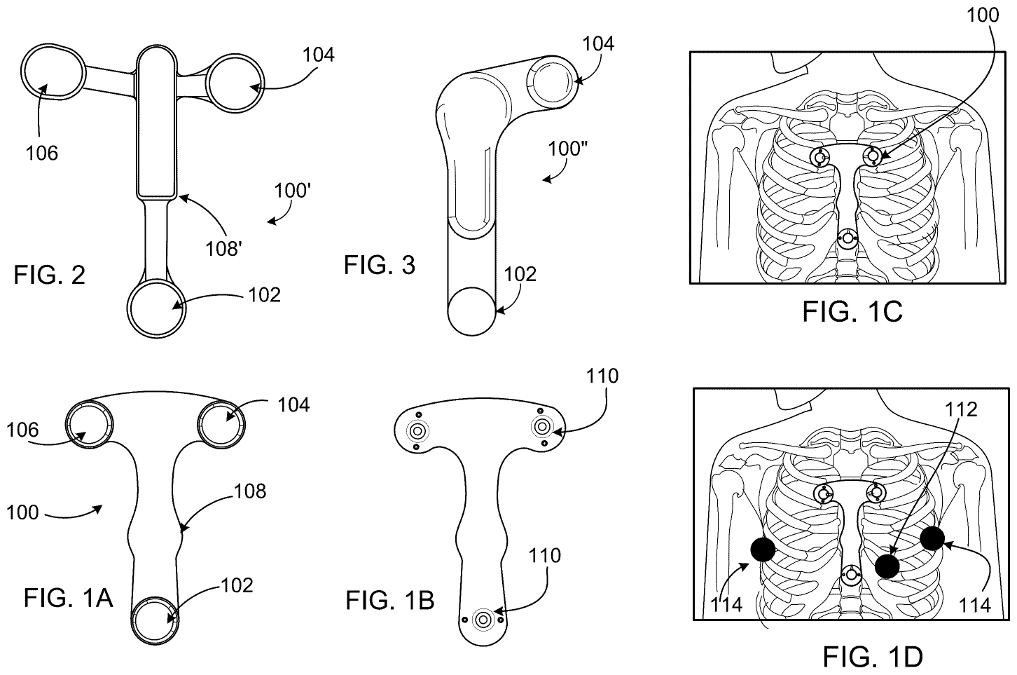

FIG. 1A illustrates a cardiac sensor 100 with multiple vibration sensors 102, 104, 106 attached by a body 108. The vibration sensors are, for example, accelerometers, microphones, or piezoelectric strain sensors, or piezoelectric ultraousound sensors that transmit and receive an ultrasonic waveform. The body 108 of the cardiac sensor 100 is a disposable material (metal or plastic). The body 108 can be non-permanently and non-invasively attached to a human subject (e.g., the chest of a user). The body 108 is generally sized so that, when worn by a user, a lower vibration sensor 102 is placed on the xiphoid (i.e., the bottom of the sternum) of the user, and upper vibration sensors 104, 106 are placed in the intercostal chest region (e.g., the second or third intercostal space, between ribs, or generally placed over the heart). The body 108 has a vertically symmetric shape so that the upper sensors 104, 106 are evenly displaced from a central axis of the body 108. The body 108 also includes attachments (e.g., snap attachments) for electrodes (e.g., electrocardiogram (ECG) electrodes) or other medical equipment (e.g., photoplethysmography (PPG) sensors). FIG. 1B illustrates a cardiac sensor 100 with snap attachments 110 placed over the vibrational sensors 102, 104, 106. The vibrational sensors of the cardiac sensor 100 detect a vibrational signature of the cardiac cycle (e.g., heart valve closure and bulk movement of the cardiac muscle) which is analyzed to predict a cardiopulmonary state of a user. FIG. 1C illustrates proper placement of the cardiac sensor 100 on a patient or user 150.

In some implementations, the vibration sensors are placed on other areas of the chest. For example, FIG. 1D indicates alternative placement of the vibro-acoustic sensors 112, 114. The vibro-acoustic sensors 112 is placed near the apex of the heart, and the vibro-acoustic sensors 114 are placed on the side of the patient's thoracic cage. The vibro-acoustic sensors 114 emphasize the pulmonary signature (e.g., lung vibro-acoustic signatures). Other vibro-acoustic sensors can be placed on other portions of the chest or body to receive other acoustic signatures.

Some implementations of these systems and methods use other cardiac sensors. For example, FIG. 2 illustrates a cardiac sensor 100′ with a body 108′ that has arms of different lengths such that the sensor 104 is closer to the center of the body than the sensor 106. In another example, FIG. 3 illustrates a cardiac sensor 100″ has only one upper vibration sensor 104.

The lower sensor 102 provides a standardized point for device placement on an individual. Additionally, placing the lower sensor 102 at the base of the rib cage allows the lower sensor 102 to receive a different vibro-acoustic signature than the upper vibration sensors. For example, a vibro-acoustic wave does not need to travel through the bony structure of the rib cage to reach the lower sensor 102. The upper sensor 104 is placed in proximity to the ascending aorta (i.e., where pressurized blood leaves the heart) and can receive strong vibro-acoustic waves, particularly as it relates to the S2 waveform. Sensors 120 and 121 (Fig D) are intended to be located over the apex (base) of the heart (sensor 120) and away from heart (sensor 121) to emphasize the pulmonary signature (lung sounds).

Cardiac sensors have different sizes (e.g., lengths and/or widths) to accommodate different users. For example, cardiac sensors come in several sizes so that a child uses a different size cardiac sensor than an adult, or so that an adult uses different sizes according to the width of his or her chest. Different sizes serve to accommodate anatomical differences in the length of the sternum from xiphoid to the 2nd or 3rd intercostal space.

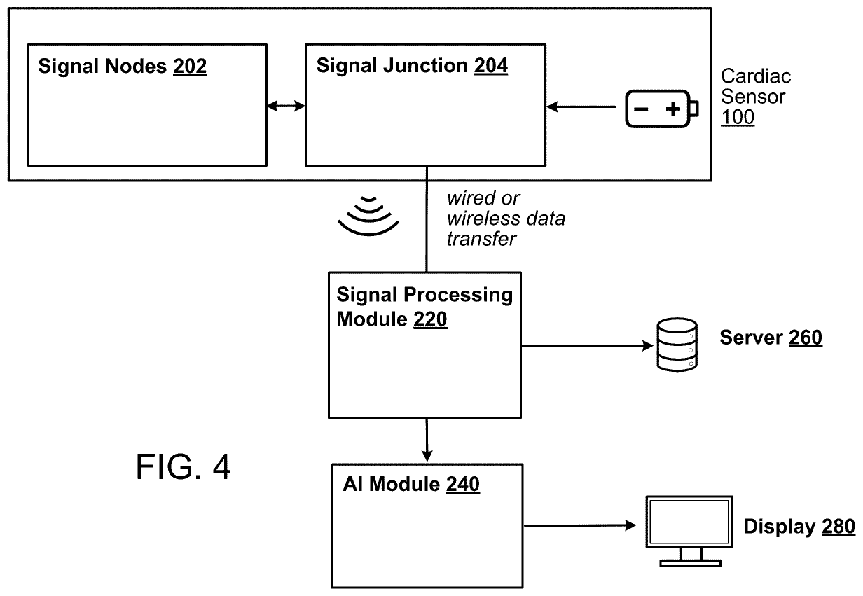

FIG. 4 illustrates a block diagram of a signal processing system 200. The signal processing system 200 can remotely processes the signals detected by a cardiac sensor 100 to reduce the electronics required within the cardiac sensor 100. For example, the signal processing system 200 includes a cardiac sensor 100, a signal processing module 220, and an artificial intelligence (AI) module 240. The cardiac sensor receives signals 202 from signal nodes (e.g., vibrational sensors, ECG sensors, PPG sensors). The cardiac sensor pre-processes the received signals 202 in a signal junction 204. For example, the signal junction 204 is a processor that converts analog signals to digital signals (or vice versa). Additionally or alternatively, the signal junction 204 can condition the signals, e.g., via voltage or current limiting, anti-aliasing filtering, and/or resampling. The signal junction 204 also manages power distribution to the sensors such that each sensor receives an appropriate supply voltage and current with minimal electrical noise. The signal junction 204 then transmits the pre-processed signals to the signal processing module 220. For example, one or more hardwire connections (e.g., serial cable, parallel cable, etc.) may be implemented. Wireless techniques such as infrared (IR), radio frequency (RF), Bluetooth, wireless Ethernet, or other electromagnetic linking techniques, may also be used. One or more protocols, transmission standards, and data formats may also be used for passing information between the signal junction 204 and the signal processing module 220.

The signal processing module 220 is a processor that fully processes the signals to extract signal features. For example, the signal processing module 220 denoises and isolate individual cardiac cycles from the signals (e.g., through Double-Density DWT Thresholding). In one embodiment, a simultaneously acquired electrocardiogram signal (“ECG”) is also acquired and used in conjunction with the sensor signals to identify individual cardiac cycles and the start of each cycle as indicated by the QRS waveform present in the ECG signal. A discrete wavelet transform is then performed on each signal. For each cardiac cycle, established physiologic knowledge indicates approximately where the waveforms of interest are expected to appear, although the desired waveforms may not be readily observable due to signal-to-noise considerations. In the wavelet domain, however, certain wavelet packets can be identified where energy is statistically elevated in regions where the targeted waveforms are expected to be found with respect to surrounding regions. By identifying the wavelet packets containing the statistically significant, locally elevated signal energy contributions and establishing a threshold for wavelet coefficients consistent with the desired signal (e.g., with respect to regions that do not contain the signal due to physiologic principles) the desired waveforms can be isolated and denoised by applying wavelet thresholding principles based on amplitude and temporal occurrence. The individual cardiac cycles is stored in a database 222. The signal processing module 220 also transmits the processed signals to the AI module 240.

The AI module 240 analyzes the denoised signals and extracts signal features that are used to predict one or more cardiovascular outcomes. Cardiovascular outcomes of interest include an estimation of blood pressure, the progression and/or severity of congestive heart failure, or an indication of the cardiovascular system's response to fluid administration. Signal features (or “predictors”) include a range of descriptive statistics and derived quantities that are calculated from the denoised signal. For example, the AI module 240 can apply machine learning algorithms to the extracted features to predict one or more cardiovascular outcomes. Additionally, the AI module can use the signals and extracted features to train machine learning algorithms or generate clinical intelligence. The AI module also initiates displays or notifications (e.g., push notifications or messages) to present the cardiovascular features to a user (e.g., through a smartphone or a monitor).

In some embodiments, all signal processing is completed with electronics within the cardiac sensor 100. For example, in some implementations, the cardiac sensor 100 has a processor that acts as a signal processing module and AI module, a memory that acts as a database, etc. In some embodiments, the cardiac sensor 100 transmits the pre-processed signals to a remote server 260 or the cloud, which acts as the signal processing module and AI module. The fully processed signals and cardiovascular features are then transmitted back to the user, e.g., on a display 280. For example, in some embodiments, a user has an app that receives his or her fully processed signals and cardiovascular features from the cloud and presents them to the user.

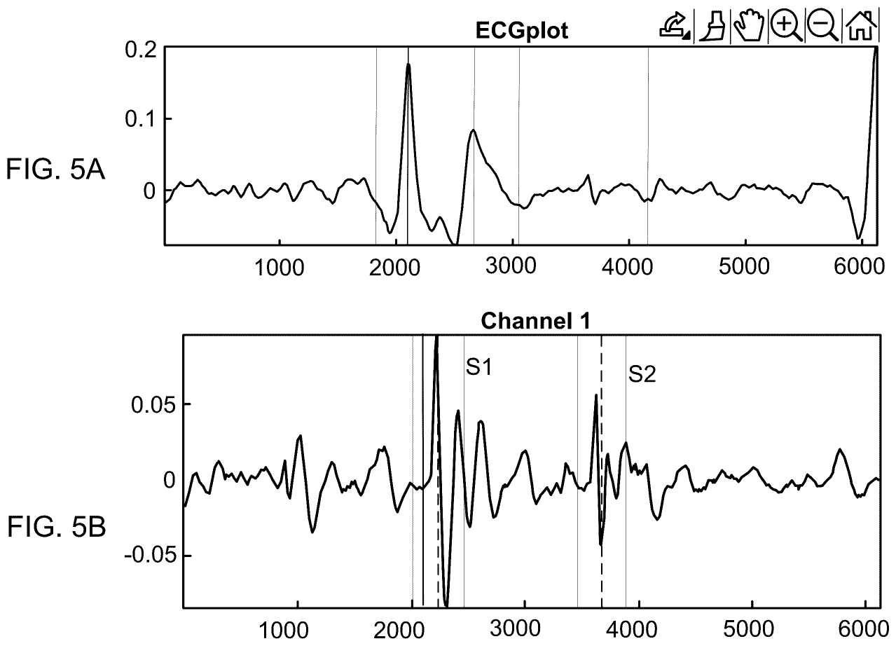

FIGS. 5A and 5B illustrate signals that are extracted from the cardiac sensor 100. For example, FIG. 5A illustrates a plot of an ECG signal that is detected by, e.g., ECG electrodes on the cardiac sensor 100. The signal processing module 220 processes the signals detected by the ECG electrodes (e.g., through a corresponding amplifier circuit) and process the signals to create the plot illustrated in FIG. 5A, which is used to predict cardiovascular features. For example, ECG signals are helpful in diagnosing abnormal heart rhythms (i.e., arrhythmias), blocked or narrowed arteries in the heart (e.g., coronary artery disease) or whether certain heart disease treatments, such as a pacemaker, are working properly. The ECG signal comprises P, Q, R, S, and T waves caused by electrical signals of the heart. The sinoatrial node (SA) is the pacemaker of the heart and produces the P wave. The QRS wave is produced by the atrioventricular node (AV).

The P wave in an ECG complex indicates atrial depolarization. The QRS is responsible for ventricular depolarization and the T wave is ventricular repolarization. FIG. 5B illustrates a plot of cardiovascular vibro-acoustic signals that are detected by, e.g., vibration sensors on the cardiac sensor 100. The signal processing module 220 processes the signals detected by the vibration sensors and process the signals to create the plot illustrated in FIG. 5B, representing one form of a denoised signal from which signal features may be extracted. Two characteristic cardiovascular vibro-acoustic signatures of the heart S1, S2 are highlighted. These characteristic cardiovascular waveforms S1, S2 are the signatures of the mitral/tricuspid valves closing (S1) and the aortic/pulmonic valves closing (S2). Cardiovascular vibro-acoustic signatures are used to detect issues that may not be detected by ECG electrodes. For example, cardiovascular vibro-acoustic signatures are helpful in diagnosing a variety of cardiovascular features, including arrhythmias, heart disease, heart failure, or blood pressure. In some embodiments, the ECG signals and the cardiovascular vibro-acoustic signatures are both utilized to comprehend both the electrical activity (e.g., the ECG signals) and the mechanical activity (e.g., the vibro-acoustic signatures) of the cardiac cycle.

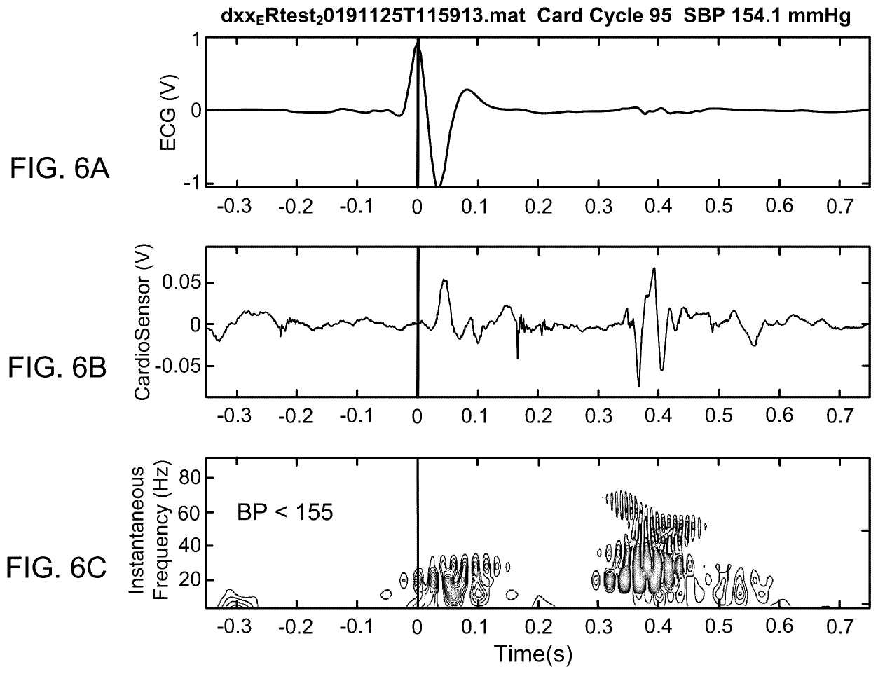

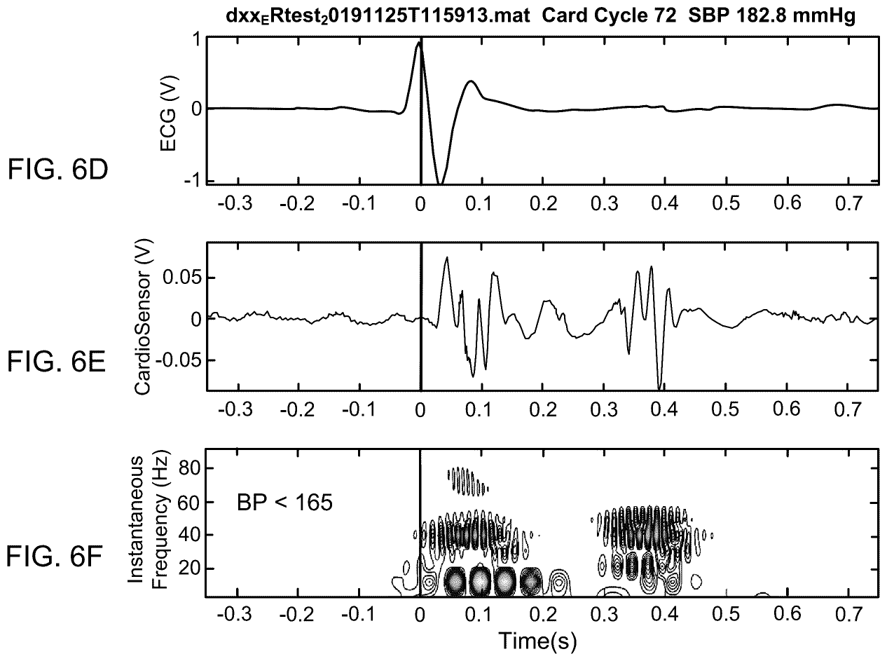

FIGS. 6A-6F illustrate diagnostic differences between ECG signals and cardiovascular vibro-acoustic signatures. FIGS. 6A-6C illustrate plots of signals from a patient with a systolic blood pressure of 154.1 mmHg for a single isolated cardiac cycle, and FIGS. 6D-6F illustrate plots of signals for another cardiac cycle from the same patient when the systolic blood pressure was measured at 182.6 mmHg. FIGS. 6A and 6D illustrate the respective ECG signals and are substantially similar, whereas and the difference in blood pressure between the two cardiac cycles is substantially different. FIGS. 6B and 6E illustrate cardiovascular vibro-acoustic signatures detected in each patient. In contrast to the ECG signals, the cardiovascular vibro-acoustic signatures show significant differences between the two observations. For example, the cardiovascular vibro-acoustic signatures in FIG. 6E (i.e., the cardiovascular vibro-acoustic signatures of the event with a systolic blood pressure of 182.6 mmHg) change much more drastically than the cardiovascular vibro-acoustic signatures in FIG. 6B (i.e., the cardiovascular vibro-acoustic signatures of the event with a systolic blood pressure of 154.1 mmHg). In some implementations, further analysis is also done on the cardiovascular signatures. For example, FIGS. 6C and 6F illustrate wavelet deconstructions into wavelet packets of the signals from cardiovascular vibro-acoustic signatures. Wavelet deconstructions are utilized for a time-frequency resolution of events. For example, periodic bursts (e.g., flutters) of high frequency content are detected as valves of the heart (e.g., the mitral valve and tricuspid valve) close. For example, maximum overlap wavelet transforms provide a high resolution time-frequency characterization of the signature of valve flutter upon closure of the valves. This approach can employ different families of wavelet filters depending on the characteristics of the specific signal including, for example, the Daubechies and Fejér-Korovkin wavelet families. These periodic bursts of energy into high frequency content (e.g., about 20 Hz to 200 Hz) in the wavelet packets suggest that the closing of the valve is detected through cardiovascular vibro-acoustic signatures. According to the plotted wavelet packets, the event with higher blood pressure has a much higher trace of frequency flutters (as illustrated in FIG. 6F) than the event with lower blood pressure (as illustrated in FIG. 6C). Additionally, the period of the frequency flutters in the event with higher blood pressure is shifted compared to the frequency flutters of the event with lower blood pressure. The event with higher blood pressure has an average flutter period of about 16.4 ms (61 Hz) and the event with lower blood pressure has an average flutter period of about 22.1 ms (45.3 Hz). The average flutter period is therefore also indicative of blood pressure, which was not reflected in ECG signals, as described above.

These differences are indicative that analyzing cardiovascular signatures is an efficient way to predict and diagnose cardiovascular outcomes that are difficult to detect through other methods (e.g., ECG signals).

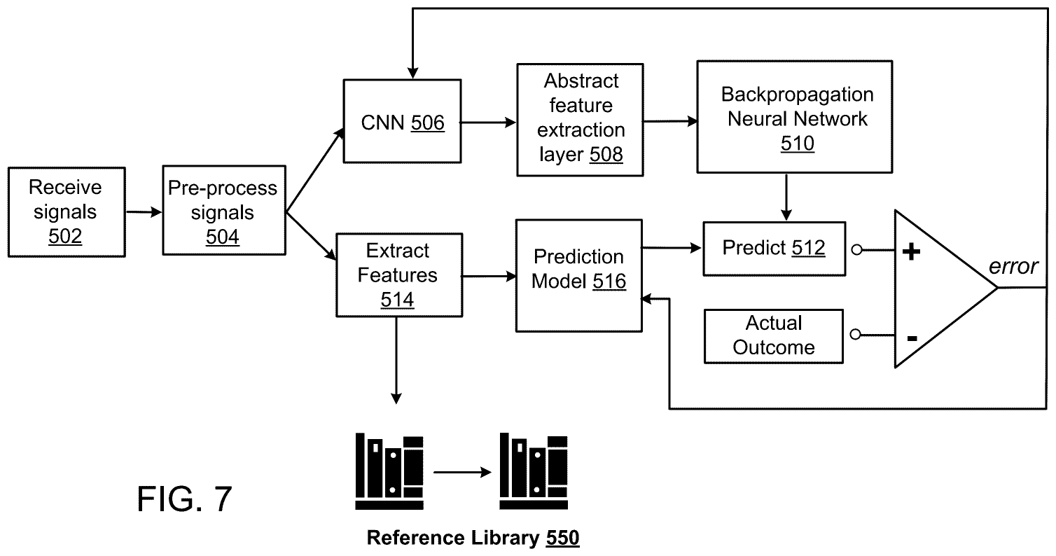

FIG. 7 illustrates a block diagram of a model architecture 500 for training a model that analyzes the fully processed signals to predict one or more cardiovascular features. For example, the model architecture 500 is used to train the AI module 240 of FIG. 4. The model architecture is built using the data from a single reference subject so that the resulting model predicts cardiovascular outcomes using features that are most relevant to that specific subject. This is less generalized than a model that is built off a number of subjects, and gives more accurate predictions for the specific subject or subjects very similar to the specific subject. First, the model architecture involves receiving signals (502) from a reference subject. For example, the signals are received by a cardiac sensor 100. Next, the model architecture involves pre-processing and denoising the received signals (504). For example, pre-processing and denoising the signals is done by a cardiac sensor 100 or by a signal processing module 220.

The pre-processed and denoised signals are then analyzed through multiple paths, which are done independently or together. In some implementations, the multiple paths are sequentially executed in repetitive passes through the flow chart 500. In other implementations, the multiple paths may be executed together simultaneously. For example, a dedicated processor or processing engine may be assigned to each path and independently execute the operations of flow chart 500. Processing sharing and multitasking techniques may also be implemented for simultaneous execution of the operations of flow chart 500.

In a first path, the pre-processed and denoised signals are used to train a deep learning convolutional neural network (CNN) (506). The CNN extracts abstract features (508) which are imperceptible to humans. Based on the training data, the CNN automatically extracts abstract features that will later be used for object classification. CNN's apply multiple temporal and spatial filters to a one dimensional or two dimensional signal to highlight, and subsequently identify, patterns or signatures in a local field of a signal that have a statistical correlation with a targeted outcome. These extracted abstract features are input into a backpropagation neural network (510), which predicts a cardiovascular outcome, such as blood pressure (512). The backpropagation neural network propagates the total loss from the abstract features back into the neural network to determine how much loss each individual node is responsible for, and subsequently updates the weights of weighted features in such a way that minimizes the total loss. For example, weights are updated by giving the nodes with higher error rates lower weights and vice versa. Once the weights are updated to minimize the total loss, the backpropagation neural network predicts a cardiovascular outcome, such as blood pressure (512). Because the actual cardiovascular outcome is known during the training phase of model development, the predicted cardiovascular outcomes are compared to the actual cardiovascular outcome to determine the prediction error. The error between the predicted and actual cardiovascular outcomes are provided as feedback to the CNN, and the process runs again to minimize the error between the predicted and actual cardiovascular outcomes. This process repeats in a cycle until the resulting model is able to correctly predict cardiovascular outcomes or a cardiopulmonary state of the reference subject.

Although the classification model is described as a convolutional neural network, in some implementations other classification models are used to relate the extracts features to the cardiovascular outcomes. For example, in some implementations, random forest models or support vector machine models are used.

In a second path, the pre-processed and denoised signals are analyzed to extract features (514). For example, the features are patterns that are recognizable, such as patterns in the signals illustrated in FIGS. 5A, 5B, and 6A-6E (e.g., patterns in cardiovascular vibro-acoustic signatures or flutter period and frequency). These extracted features are stored as a signal phenotype in a reference library 550, and are later used to compare features between subjects. The extracted features are also input into a prediction model (e.g., a random forest regression model, a neural network, or a support vector machine) (516) which predicts cardiovascular outcomes (512) based on the extracted features. For example, flutter period is used to predict blood pressure, as described above. In some embodiments, the prediction model utilizes the extracted features along with abstract features extracted by a CNN. Because the actual cardiovascular outcome is known, the predicted cardiovascular outcome is compared to the actual cardiovascular outcome to determine whether the prediction error. The error between the predicted and actual cardiovascular outcome is provided as feedback to the prediction model, and the process begins again in a loop until the resulting model is able to correctly predict cardiovascular outcomes of the reference subject. After the prediction model correctly predicts the cardiovascular features (e.g., with at least 95% accuracy), the prediction model is stored in the reference library 550. The prediction model is linked to its signal phenotype or extracted features that were previously stored in the reference library 550.

Some applications are implemented with only one of these paths.

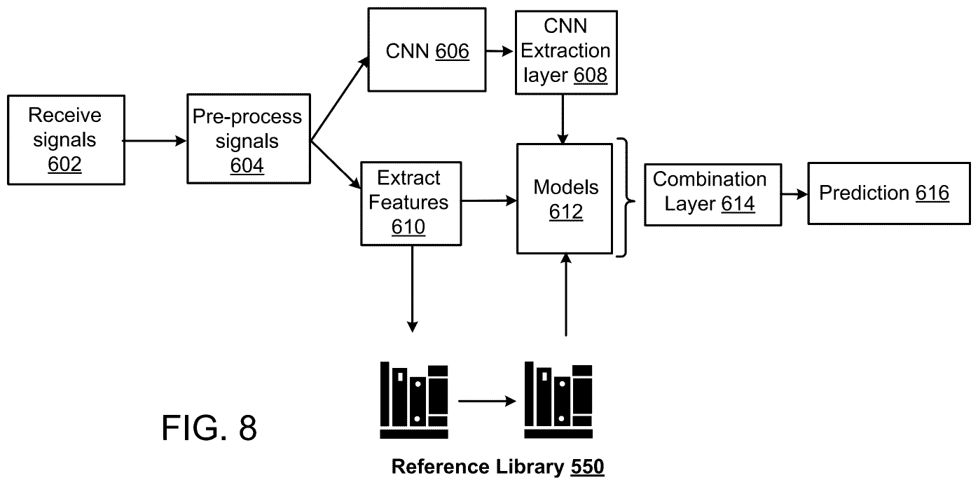

FIG. 8 illustrates a block diagram of a model architecture for analyzing signatures of a new subject. For example, the model trained from the model architecture 500 is used to analyze signatures of a new subject. First, the model receives signals from a new subject (602). For example, the signals are received by vibration sensors of a cardiac sensor 100. The signals are denoised and pre-processed (604). For example, the signals are denoised and pre-processed by a cardiac sensor 100 or by a signal processing module 220. The denoised and pre-processed signals are input into a convolutional neural network (606) to extract abstract features (608) in a CNN extraction layer. Additionally or alternatively, features of patterns that are recognizable are extracted (610) to determine the new subject's signal phenotype and to locate similar subjects in the reference library 550. The reference library 550 stores subject features (e.g., signal phenotypes) and corresponding prediction models (e.g., random forest models, neural networks, or support vector machines) that were analyzed by the model architecture 500. Once a set of subjects that are similar to the new subject (e.g., a set of subjects with a similar signal phenotype) is located, a corresponding set of prediction models is be pulled from the reference library 550. The features of patterns, alone or along with abstract features extracted by the CNN, are input into the set of prediction models (612), which each individually predict cardiovascular outcomes. The individual predictions are combined (614) in a combination layer to aggregate the predictions into a single prediction (616) of the cardiovascular outcome. For example, the individual predictions are averaged to aggregate a number of predictions into a single prediction. In another example, individual predictions are weighed by the relative similarity of each model to the new subject's signal phenotype.

This specification describes devices, methods, and systems for sensing cardiac vibro-acoustic signals. It will be appreciated that various changes may be made by those skilled in the art without departing from the spirit and scope of this disclosure.

What is claimed is:

1. An apparatus comprising:
a body that is attachable non-permanently and non-invasively to a human subject's chest;
at least two vibration sensing elements contained in the body, wherein the vibration sensing elements measure a vibrational signature on a chest wall of the subject, wherein a first vibration sensing element is indexed from a xiphoid of the subject and a second vibration sensing element is located in or between a second and third intercostal space of the subject; and
at least two electrodes contained within the body, the at least two electrodes electrically attachable to skin of the subject and a corresponding amplifier circuit to acquire an ECG signal;
wherein the apparatus is configured to synchronously acquire signals from the chest wall of the subject and transmit said signals to an external data collection device;
wherein an external data collection device monitors the signals, wherein the signals are indicative of a hemodynamic state of the subject and provide diagnostic decision support related to one or more of: blood pressure, heart valve function, acute cardiopulmonary system failure, and chronic heart conditions.

2. The apparatus of claim 1, wherein acute cardiopulmonary system failure comprises one or more of myocardial infarction, congestive heart failure, or cardiopulmonary collapse.

3. The apparatus of claim 1 or claim 2, wherein chronic heart conditions comprise hypertension.

4. The apparatus of any one of claims 1-3, further comprising a third vibration sensing element located in or between the second and third intercostal space of the subject.

5. The apparatus of any one of claims 1-4, wherein the body comprises multiple arms, each arm having a different length, wherein one of the vibration sensing elements is contained within each arm.

6. A method of assessing cardiac activity, the method comprising:
isolating components of the vibrational signature which are attributable to heart valve closure and bulk movement of a heart during each cardiac cycle, with reference to features of the ECG signal, such that mechanical and electrical activity of the cardiac cycle is simultaneously comprehended, and such that signal features indicative of cardiopulmonary state are extracted from the combined signal set, wherein extracted signal features and methods include one or more of:
timing, duration, and intensity of vibroacoustic cardiac signals relative to each other and ECG waveform features,
frequency content of vibroacoustic cardiac signals in a 10 Hertz to 200 Hertz frequency range,
energy levels in a subset of wavelet packets determined uniquely for each subject based on signals of the subject, which are combined with observations from other subjects and states with similar attributes to comprise a signal phenotype,
a time of arrival of signal wavelet packets at each sensor, the wavelets being from separate heart signatures, to normalize for signal transfer function between source and receiver that is different for each individual, and
high resolution time-frequency characterization of heart signatures using maximum overlap wavelet transforms such that the signature of valve flutter upon closure is characterized.

7. The method for operating on the processed signals and signal features from claim 6, the method comprising:
training individual models for single individuals in a training set over short periods of time to create a reference library of models associated with a signal phenotype; and
combining individual reference models with an abstract feature extracted from a multi-layer convolutional neural network as inputs to a prediction model;
wherein new observations from previously unseen subjects are compared to pre-existing signal phenotypes to select a subset of reference models from the reference library that best match the new observations, such that a new prediction is made by presenting new data to a selected subset of reference models and determining a consensus output weighted by a relative similarity of each model to the new signal phenotype.

8. The method of claim 7, wherein the model is a random forest, neural network, or support vector machine model.

9. The method of any one of claims 6-8, wherein the method further comprises receiving the components of the vibrational signature which are attributable to heart valve closure and bulk movement of the heart from at least two vibration sensing elements, wherein the vibration sensing elements measure a vibrational signature on a chest wall of a subject, wherein a first vibration sensing element is indexed from a xiphoid of the subject and a second vibration sensing element is located in or between a second and third intercostal space of the subject.

10. The method of claim 9, further comprising receiving the components of the vibrational signature which are attributable to heart valve closure and bulk movement of the heart from at least a third vibration sensing element, the third vibration sensing element located in or between the second and third intercostal space of the subject.

11. The method of any one of claims 6-10, further comprising receiving the ECG waveform features from at least two electrodes electrically attachable to skin of the subject and a corresponding amplifier circuit to acquire the ECG waveform features.
